(19) United States
(12) Patent Application Publication
(10) Pub. No.: US 2015/0173709 A1
(43) Pub. Date: Jun. 25, 2015

(54) ULTRASOUND PROBE CENTERING DEVICE

(71) Applicant: CIVCO MEDICAL INSTRUMENT CO., INC., Kalona, IA (US)

(72) Inventor: Willet F. Whitmore, III, Longboat Key, FL (US)

(21) Appl. No.: 14/384,773

(22) PCT Filed: Mar. 13, 2013
(86) PCT No.: PCT/US2013/030756

§ 371 Date: Sep. 12, 2014

Related U.S. Application Data
(60) Provisional application No. 61/611,228, filed on Mar. 15, 2012.

(51) Int. Cl. A61B 8/00; A61B 8/12

(52) U.S. Cl. CPC A61B 8/4281; A61B 8/12

(57) ABSTRACT

A centering device for use with an ultrasound probe and the combination of that device and the probe is disclosed. The probe has a distal portion in which an ultrasound emitter is located. The emitter is arranged to be rotated about the central longitudinal axis of the probe without rotating the probe itself to provide ultrasound images of a patient extending 360 degrees about that axis. The centering device includes an inner sheath and an outer sheath surrounding the inner sheath to form a space therebetween. The space is inflatable by a liquid to expand to form a cylindrical annularly shaped chamber surrounding the distal portion of the probe, thereby centering the distal portion of the probe within the patient.

### CROSS-REFERENCE TO RELATED APPLICATIONS

This PCT application claims the benefit under 35 U.S.C. §119(e) of U.S. Provisional Patent Application Ser. No. 61/611,228, filed on Mar. 15, 2012, entitled ULTRASOUND PROBE CENTERING DEVICE whose disclosure is incorporated herein by reference.

### BACKGROUND OF THE INVENTION

This invention relates to medical devices and more particularly to ultrasound probes and devices for centering the same within a cavity or lumen in the body of a patient.

Single sector ultrasound probes have been available for some time for providing imaging of a cavity or lumen, e.g., the rectum, in a patient. As is known such probes make use of an ultrasound emitter that is directed radially outward from the probe's distal end portion in a fixed direction. Thus, the use such a single sector probe to accomplish 360 degree imaging of the rectum of a patient requires the imaging of one circumferential portion of the rectum wall, then rotation of the probe within the rectum so that the probe's emitter is directed to another circumferential portion of the rectum wall, and so forth until the full 360 imaging has been achieved.

Electronic ultrasonic probes are available today which include a distally located working head comprising an ultrasound emitter, e.g., a linear array of ultrasound crystals. The emitter is arranged to be rotated about the central longitudinal axis of the probe so as to provide a full 360 degree image without necessitating rotating the probe itself. The use of such probes to provide good 360 degree images within a lumen or cavity, e.g., the rectum, of a patient presents several issues. For example, if the probe is disposed within the rectum but isn't centered, e.g., is contacting one portion of the wall of the rectum, while the diametrically located portion of the probe is spaced from the wall of the rectum good imaging of all 360 degree portions of the patient's rectum adjacent the probe's ultrasound emitter will not be possible, i.e., some portions of the patient's rectum and the tissue behind it will not be adequately imaged since such portions will lie outside of the range of optimized resolution of the probe for the particular ultrasound frequency utilized.

Heretofore fluid filled balloon interfaces have been utilized to effect a desired positioning of the working head of an ultrasonic probe within the body of a patient. However, such inflatable prior art devices allow for the working head of the ultrasonic probe to float freely within the fluid interface space, thereby inherently presenting the problem of unreliable interface maintenance. Solid (e.g., silicone) interfaces have been used and are available to mitigate this problem, but such interfaces are difficult to use in clinical applications where a circumferential interface is needed. This is especially true for use in the rectum where it is desirable to have as small a diameter as possible when inserting and removing the device (passing through the anal sphincter) for reasons of comfort and safety.

In my prior U.S. Pat. Nos. 6,932,771 and 7,563,260, which are assigned to the same assignee as this invention, there are disclosed asymmetric fluid filled balloon standoffs for use with a single sector ultrasound probe to hold and maintain the probe at any desired angular position within the patient's body to maintain reliable tissue separation. While the devices constructed in accordance with the teachings of my aforementioned patents are suitable for their intended uses, they are not suitable for use with a probe providing a full 360 degree view without rotating the probe.

Thus, a need exists for a device that can be used with such a probe to achieve good 360 degree imaging. The present invention addresses that need by providing a centering device for the working end portion of a 360 degree ultrasonic probe.

### SUMMARY OF THE INVENTION

One aspect of this invention is a centering device for use with an ultrasound probe. Another aspect of this invention comprises the combination of the ultrasound probe and the centering device.

The ultrasound probe has a distal portion including a working head in the form of ultrasound emitter, e.g., a linear array of ultrasound crystals, located within the distal portion of the probe. The distal portion of the probe has a central longitudinal axis and is arranged for insertion into a lumen or cavity, e.g., the rectum, of a patient to provide ultrasound images of the patient extending 360 degrees about the central longitudinal axis of the probe without necessitating rotating the probe about that axis.

The centering device basically comprises an inner sheath and an outer sheath. Each of said sheaths is a cylindrical member (e.g., a flexible, condom-like member). The inner sheath is disposed within the outer sheath to faun a space therebetween. The centering device is arranged for disposition on the distal portion of the ultrasound probe. The space between the sheaths is inflatable by a fluid (e.g., water) to cause the outer sheath to act like a balloon, i.e., move radially outward, to form a cylindrical shaped annular fluid filled chamber surrounding the distal portion of the probe to center the distal portion of the probe within the lumen or cavity of the patient.

In accordance with one preferred aspect of this invention the outer sheath and the inner sheath are each formed of a resilient material, e.g., latex, polyurethane, etc., and each includes a closed distal end. The closed distal ends of the two sheaths are secured (e.g., welded) together at a joint immediately adjacent the longitudinal axis of the probe. In addition, the centering device additionally includes an inlet port in fluid communication with the space between the sheaths and an outlet port also in fluid communication with the space between the sheaths. The inlet port serves to effect the inflation of the annular fluid filled chamber with a liquid (e.g., water), while the outlet port serve to effect the removal of air from the annular fluid filled chamber.

### DESCRIPTION OF THE DRAWING

FIG. 1 is a sectional view of one preferred exemplary embodiment of a centering device constructed in accordance with this invention;

FIG. 2 is a sectional view, like that of FIG. 1, but showing another preferred exemplary embodiment of a centering device constructed in accordance with this invention;

FIG. 3 is a sectional view showing the centering device of FIG. 1 mounted on the distal end portion of a 360 degree imaging ultrasonic probe;

FIG. 4 is a sectional view showing the centering device of FIG. 1 mounted on the distal end portion of a 360 degree imaging probe, with the centering device being inflated to center the probe within a lumen or cavity, e.g., the rectum, in a patient (the lumen or cavity not being shown in this figure); and

FIG. 5 is an enlarged sectional view taken along line 5-5 of FIG. 4, but showing the lumen or cavity in which the probe is centered by the centering device of FIG. 1.

### DETAILED DESCRIPTION OF THE PREFERRED EMBODIMENT

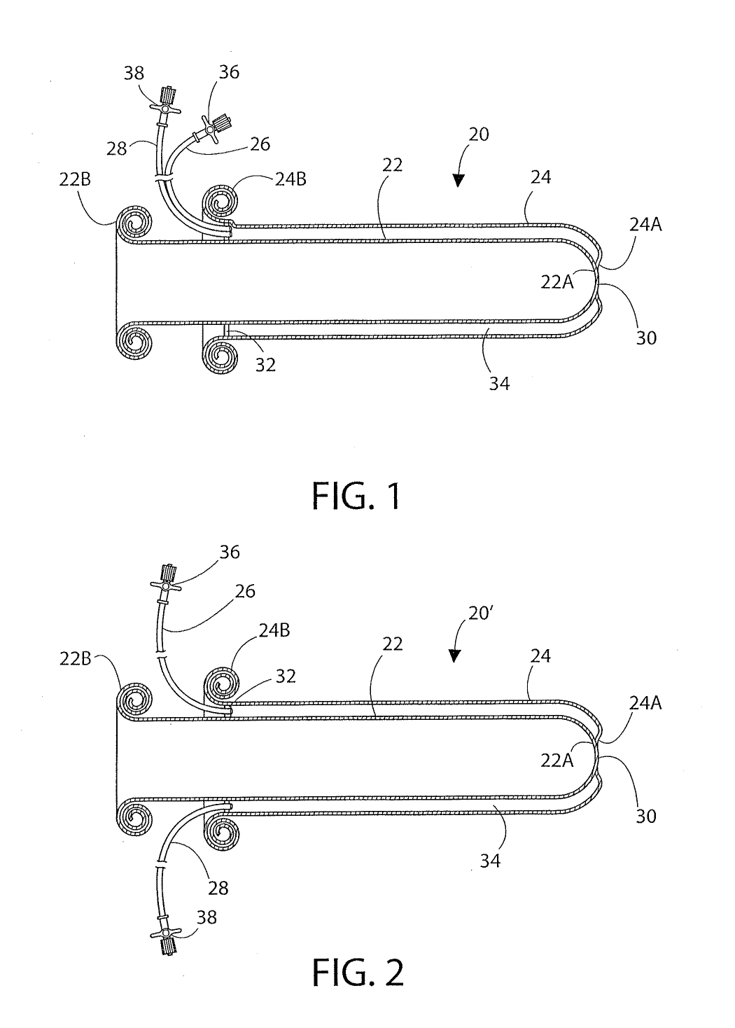

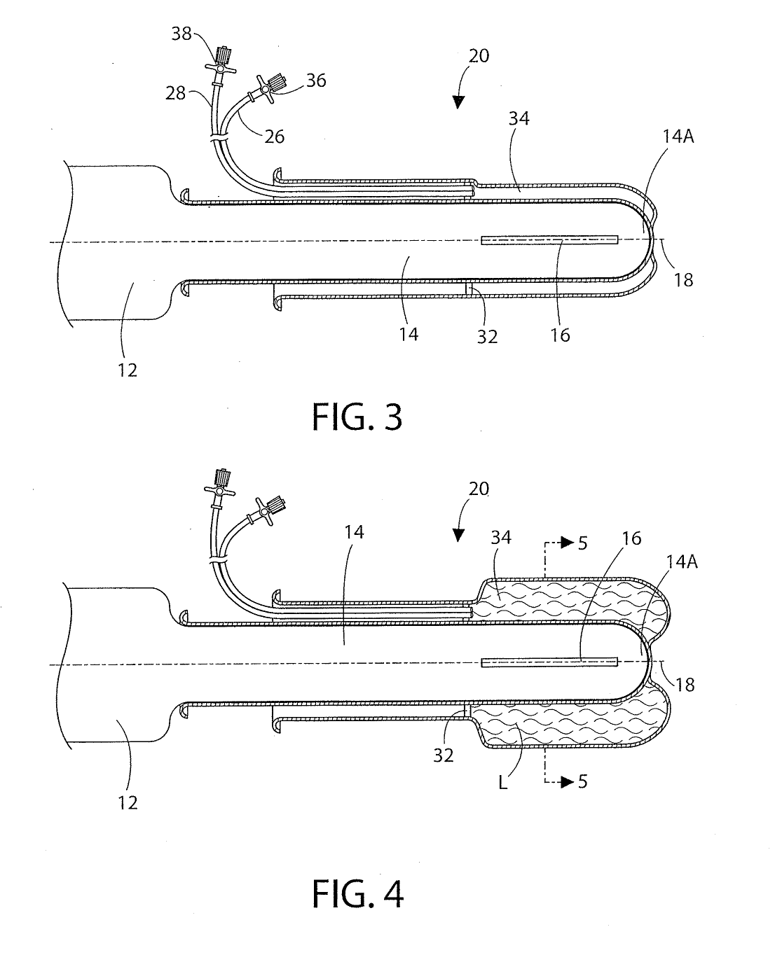

Referring now to the various figures of the drawing wherein like reference characters refer to like parts, there is shown at 20 in FIG. 1 one exemplary embodiment of a centering device constructed in accordance with this invention for use with a 360 degree imaging ultrasonic probe 10. A variant centering device 20′ is shown in FIG. 2. The probe 10 is shown in FIGS. 3-5 and can be of any construction. To that end, as shown in FIG. 3, the probe 10 basically includes an elongated body portion 12 having a distal end portion 14. The distal end portion forms the working head of the probe, i.e., the ultrasonic emitter. That emitter may be in the form of a linear array 16 of ultrasonic crystals. The probe includes a longitudinal central axis 18. The free end of the distal end portion of the probe is designated by the reference number 14A.

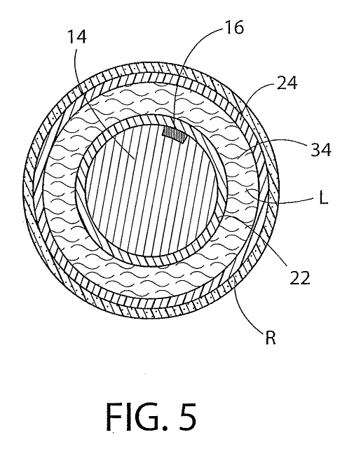

The centering device 20, will be described in detail shortly, suffice it to say that it includes an inflatable chamber or balloon which is arranged to be mounted on the distal end portion 14 of the probe 10 and thereafter inflated, e.g., filled with a liquid, such as but not limited to water, to effect the centering of the probe within any lumen or cavity, e.g., the rectum, of a patient to enable the 360 degree imaging of the patients' surrounding anatomic structures as shown in FIG. 5.

As best seen in FIG. 1 the centering device 20 basically comprises an inner sheath or condom-like member 22, an outer sheath or condom-like member 24, a filling or inflation conduit or tubing 26 and a venting conduit or tubing 28. Both of the sheaths are cylindrical members formed of a resilient flexible material, e.g., latex, polyurethane or any other suitable material. The inner sheath 22 includes a closed distal end 22A. The outer sheath is also includes a closed distal end 24A. The inner sheath 22 is disposed within the outer sheath and their two closed distal ends 22A and 24A are secured, e.g., welded, together, at a joint 30. The joint 30 can be formed by other means than welding, e.g., an adhesive. The proximal end of the outer sheath is open and is rolled up, like a condom, at 24B. The proximal end of the inner sheath is open and is rolled up, like a condom, at 22B. The inner sheath is secured to the outer sheath at a circumferentially extending joint 32 which is located just proximally of working head of the probe.

The space that is located between the inner and outer sheaths between the joints 30 and 32 forms a sealed, hollow chamber 34 that extends the length of the working head of the probe (as can be seen in FIGS. 3 and 4). The chamber 34 is arranged to be inflated with a liquid L, e.g., water, as will be described later, whereupon the outer sheath 24 expands or otherwise moves outward like a balloon to form an elongated cylindrically shaped (e.g., sausage-shaped) body whose outside diameter is substantially constant along its length as shown in FIGS. 4 and 5.

The inflation of the chamber 34 is effected via the conduit or tubing 26. To that end the distal end of the tubing 26 extends through the joint 32 and is in fluid communication with the chamber 34. The proximal end of the tubing 26 is in the form of a stop-cock 36 which is arranged to be connected to a source of liquid, e.g., water (not shown), so that when the stop cock 36 is opened the liquid can be introduced through the conduit 26 into the chamber 34 to inflate it. As mentioned earlier, the device 20 also includes a vent conduit or tubing 28. The distal end of that tubing also extends through the joint 32 and is in fluid communication with the chamber 34. The proximal end of the tubing 28 is in the form of a stop-cock 38 which when opened is arranged to allow any air within the chamber 34 to pass through the conduit 28 and the opened stop-cock 38 to vent to the ambient atmosphere. The conduits 26 and 28 can be located at the same portion of the joint 32, such as shown in the embodiment 20 of FIG. 1, or may be located at diametrically opposed portions of the joint 32, such as shown in the embodiment 20′ of FIG. 2.

The mounting of the centering device 20 onto the probe 10 is achieved by extending the distal free end of the probe 10 into the hollow interior of the inner sheath 22 while the proximal end portions 22B and 24B, respectively, of the sheaths 22 and 24 are rolled up and so that the joint 30 at the distal end of those sheaths is coaxial with the central longitudinal axis 18 of the probe 10 as shown in FIG. 3. The rolled up proximal end portion 22B of the inner sheath can then be unrolled to dispose it further proximally of the probe's working head. The conduits 26 and 28 can then be extended longitudinally along the outer surface of the now unrolled inner sheath 22, whereupon the rolled up proximal end portion 24B of the outer sheath can then be unrolled to dispose it further proximally of the probe's working head as shown in FIG. 3. With the sheaths thus unrolled, the two conduits 26 and 28 and their respective stop-cocks 36 and 38 will be readily accessible to effect the inflation of the centering device 20. Thus, once the probe is in the desired position within the patient's body, e.g., within the patient's rectum R, the stop-cock 26 can be opened to enable water or any other suitable liquid L to be introduced, e.g., pumped into, the chamber 34 to effect its inflation. At the same time or some time thereafter, the stop-cock 38 can be opened to enable any air that is present in the chamber 34 from venting to the ambient atmosphere.

With the centering device inflated as just described the outer surface of the outer sheath or balloon will be in engagement with the inner surface of the wall of the rectum R with the working head of the probe centered and with a water interface between the ultrasonic emitters and the tissue to be imaged as shown clearly in FIG. 5.

In order to keep the working head of the probe centered in the circumferential fluid filled balloon and keep the fluid interface constant for any chosen volume, the expandable chamber (the balloon formed by the outer sheath 24) is fixed in the center at the free end or tip 14A of the probe to the inner sheath 22 by joint 30, and the proximal portion of the sheath 24 closely engages the body of the probe 10 at a point proximal to the probe's imaging region or working head.

As should be appreciated from the foregoing, the centering device of the present invention allows imaging about the entire circumference of the working (imaging) head of the probe while keeping that working head centered in the cavity or lumen of the patient's body in which it is inserted. Thus, for applications involving imaging of the rectum, an electronic ultrasound probe making use of the centering device of this invention can provide a series of ultrasound images extending about the full 360 degree circumference of the probe without moving the probe. Moreover, and quite significantly the centering of the probe within the cavity being explored is achieved by providing an inflatable chamber, which when inflated is of a cylindrical shape of a substantially constant diameter along its length. This ensures that the images of the patient's rectal wall and tissue immediately behind it that are produced by the probe will be within of the range of optimized resolution of the probe for the particular ultrasound frequency utilized.

As is known the frequency range of the ultrasound transducers for medical use is typically from 2 to 20 MHz with the lower 2-5 MHz providing greater depth of tissue penetration at the cost of lower image resolution compared to 20 MHz. For example, an ultrasound probe operating at a frequency of 5 MHz provides generally good resolution at 3-5 cm fluid/tissue depth, so this is the “optimized” or best viewing distance from the surface of the transducer for that frequency range (including the acoustic window provided by the fluid interface). A probe operating at 20 MHz provides an optimized or best image at 0-2 cm because of the higher resolution that can be used to advantage when less tissue or tissue plus fluid interface is used. So, in the best use case of the present invention with such probes, only sufficient fluid as required to achieve good surface contact with the rectal wall and displacing any surrounding air (filling contact gaps) should be used and the frequency chosen or adjusted to achieve an “optimized” resolution (highest frequency) that still has sufficient fluid/tissue penetration to visualize the target tissue. For imaging of the rectal wall using a probe operating at 5 MHz with a centering device constructed in accordance with this invention one can successfully image the rectal wall by disposing the probe at approximately 3 centimeters from it. Thus, using the centering device of this invention, there should be a separation of approximately 2 centimeters from the outer surface of the probe's distal end portion 14 to the surface of the rectal wall R. The centering device 20 provides that desired separation. Moreover, the inflation of the centering device also pushes out any interposed air, thereby providing a fluid interface between the probe and the surface of the tissue to be imaged to ensure that a good image is produced.

As discussed earlier the inflation of the centering device results in the creation of an annular fluid-filled, e.g., water-filled, chamber or balloon which is cylindrical in shape and resembles a sausage, rather than assuming a bulbous, e.g., spherical or ovoid, shape. This action ensures that the working head of the ultrasonic probe is centered within the cavity of the patient along the entire length of the working head. To achieve that end, the material making up the portion of the outer sheath that is disposed over the probe's working head can be formed of varying thickness along the length of that portion of the sheath so that the central region will not bulge out, but instead stay of a substantially constant outside diameter. Alternatively, the material making up the portion of the outer sheath that is disposed over the probe's working head may be fabricated so that it provides varying resistance to stretching along its length so that when the chamber or balloon is inflated it assumes the desired true cylindrical or sausage-like shape.

As should be appreciated by those skilled in the art the subject invention solves various problems of the prior art. For example, it provides a relatively uniform and consistent thickness of fluid interface between the ultrasound crystals/imaging device surface and the tissue being imaged for a 360 degree imaging transducer regardless of transducer position within a patient (e.g., even when it is pressed against the tissues being viewed). In addition, it provides an adjustable thickness of the fluid interface. Further still, it serves as a better device for eliminating air bubbles in the fluid interface. In particular, the present invention provides a novel tubing or conduit arrangement that allows fluid filling of its inflatable chamber while either simultaneous or delayed venting of air bubbles occur through a second conduit or tubing distinct from the filling conduit tubing. The result of the second conduit acting as an air vent permits a significant reduction in the normally required multiple syringe manipulations (that is a characteristic of all prior art devices used for this purpose) to achieve an air bubble free fluid filled balloon.

Without further elaboration the foregoing will so fully illustrate my invention that others may, by applying current or future knowledge, adopt the same for use under various conditions of service.

I claim:

1. A centering device for use with an ultrasound probe, the ultrasound probe having a distal portion and an ultrasound emitter located within the distal portion of the probe, the distal portion of the probe having a central longitudinal axis and being arranged for insertion into a cavity in the body of a patient to provide ultrasound images of the patient extending 360 degrees about the central longitudinal axis without rotation of the probe about the central longitudinal axis, said centering device comprising an inner sheath and an outer sheath, each of said sheaths being a cylindrical member, with said inner sheath being disposed within said outer sheath to form a space therebetween, said device being arranged for disposition on the distal portion of the probe, said space between said sheaths being inflatable by a fluid to cause said outer sheath to move radially outward to form a cylindrical annular fluid filled chamber surrounding the distal portion of the probe to center the distal portion of the probe within the cavity of the patient.

2. The centering device of claim 1 wherein said outer sheath and said inner sheath each include a closed distal end, and wherein said closed distal end of said sheaths are secured together at a joint immediately adjacent the central longitudinal axis.

3. The centering device of claim 2 wherein said inner sheath is formed of a resilient material.

4. The centering device of claim 2 wherein said outer sheath is formed of a resilient material.

5. The centering device of claim 3 wherein said outer sheath is formed of a resilient material.

6. The centering device of claim 1 wherein said centering device additionally comprises an inlet port in fluid communication with said space between said sheaths, said inlet port being arranged to effect the inflation of said annular fluid filled chamber with a liquid.

7. The centering device of claim 6 wherein said centering device additionally comprises an outlet port in fluid communication with said space between said sheaths, said outlet port being arranged to effect the removal of air from said annular fluid filled chamber.

8. The centering device of claim 1 wherein the distal portion of the probe is of cylindrical shape and includes a free end and wherein said sheaths of said centering device include portions that are arranged to be rolled up and unrolled, and wherein said centering device is arranged to be applied to the free end of the probe with said rolled up portion of said sheaths rolled up and once so applied to the free end of the probe said rolled up portions of said sheaths are adapted to be unrolled to cover contiguous distal portion of the probe.

9. The centering device of claim 2 wherein said sheaths are welded together at said joint.

10. In combination an ultrasound probe and a centering device, said ultrasound probe having a distal portion and an ultrasound emitter located within said distal portion of said probe, said distal portion of the probe having a central longitudinal axis and being arranged for insertion into a cavity in the body of a patient to provide ultrasound images of the patient extending 360 degrees about said central longitudinal axis without rotation of said probe about said central longitudinal axis, said centering device comprising an inner sheath and an outer sheath, each of said sheaths being a cylindrical member, with said inner sheath being disposed within said outer sheath to form a space therebetween, said device being disposed on the distal portion of said probe, said space between said sheaths being inflatable by a fluid to cause said outer sheath to move radially outward to form an form a cylindrical annular fluid filled chamber surrounding the distal portion of said probe to center said distal portion of said probe within the cavity of the patient.

11. The combination of claim 10 wherein said outer sheath and said inner sheath each include a closed distal end, and wherein said closed distal end of said sheaths are secured together at a joint immediately adjacent said central longitudinal axis.

12. The combination of claim 11 wherein said outer sheath is formed of a resilient material.

13. The combination of claim 12 wherein said outer sheath is formed of a resilient material.

14. The combination of claim 10 wherein said centering device additionally comprises an inlet port in fluid communication with said space between said sheaths, said inlet port being arranged to effect the inflation of said annular fluid filled chamber.

15. The combination of claim 10 wherein said centering device additionally comprises an outlet port in fluid communication with said space between said sheaths, said inlet port being arranged to effect the removal of air from said annular fluid filled chamber.

16. The combination of claim 14 wherein said centering device additionally comprises an outlet port in fluid communication with said space between said sheaths, said inlet port being arranged to effect the removal of air from said annular fluid filled chamber.

17. The combination of claim 10 wherein said distal portion of said probe is of cylindrical shape and includes a free end and wherein portions of said sheaths of said centering device are arranged to be rolled up and unrolled, and wherein said centering device is arranged to be applied to said free end of said probe with portions of said sheaths rolled up and once so applied to said free end of said probe said rolled up portions of said sheaths are adapted to be unrolled to cover contiguous distal portions of said probe.

18. The combination of claim 11 wherein said sheaths are welded together at said joint.

19. The combination of claim 10 wherein said ultrasound emitter comprises a linear array of ultrasound crystals.
